(19) United States
(12) Patent Application Publication
(10) Pub. No.: US 2017/0224756 A1
(43) Pub. Date: Aug. 10, 2017

(54) Antioxidants Against Oxidative Stress During a Stressor Event

(71) Applicant: MERIAL INC., Duluth, GA (US)

(72) Inventors: Alphons Rutten, Avrille (FR); Caroline Pommellet, Carantec (FR); Pierre Chicoteau, Avrille (FR)

(73) Assignee: MERIAL INC., Duluth, GA (US)

(21) Appl. No.: 15/423,280

(22) Filed: Feb. 2, 2017

Related U.S. Application Data
(60) Provisional application No. 62/292,192, filed on Feb. 5, 2016.

(51) Int. Cl. A61K 36/87; A61K 9/00; A61K 47/02; A23K 20/10; A61K 39/215; A61K 39/12; C12N 7/00; A23K 20/20; A61K 33/04; A61K 47/12

(52) U.S. Cl. CPC A61K 36/87; A61K 33/04; A61K 9/0095; A61K 47/02; A61K 47/12; A61K 39/215; A61K 39/12; C12N 7/00; A23K 20/30; A23K 20/10; A61K 2039/552

(57) ABSTRACT

The present invention encompasses an effervescent Grape extract plus selenium composition which improves stress response. The invention also encompasses methods of improving stress response, such as stress response to vaccination or weaning, by administration of the composition. The invention also encompasses methods of producing meat with improved quality by raising an animal on a diet supplemented with an effervescent Grape extract plus selenium composition.

### CROSS REFERENCE TO RELATED APPLICATION

This application claims priority to provisional application U.S. Ser. No. 62/292,192, filed on 5 Feb. 2016, and incorporated by reference herein in its entirety.

### FIELD OF THE INVENTION

The present disclosure relates generally to grape extract and selenium antioxidant compositions and methods of using the compositions in animals for improving response to stress and improving meat quality.

### BACKGROUND

Antigenic challenge, including vaccination and subsequent immune response, induces oxidative stress in both humans and animals. For example, vaccination induces a cytokine response and a fall in antioxidant status in humans. Inflammation caused by typhoid vaccination in human forearms caused widespread endothelial dysfunction, reduced vascular nitric oxide bioavailability and increased oxidative stress. These actions are partially reversible with locally administered ascorbic acid. These findings suggest a role for reactive oxygen species in inflammation-induced endothelial dysfunction (Clapp et al, 2004).

As another example, the chicken's immune response whether induced by natural infection by microbes or challenge by vaccine, also induces oxidative stress. Keles et al. (2010) reported that natural Marek's Disease infection increased DNA, lipid, and protein damage in the peripheral plasma of White-Lohmann hens compared to control hens, while the total antioxidant activity and GSH level also decreased. Costantini and Dell'Omo (2006) also showed that immune challenge like phytohaemagglutinin skin test, which stimulates a T-cell-mediated immune response, stimulates the plasma reactive oxygen metabolite levels, and reduced the total plasma antioxidant as well. These observations are not surprising. Various inflammatory cells, like macrophages and neutrophils, generate considerable reactive oxygen species to kill invading microorganisms when activated. These reactive oxygen species produced during the phagocytosis process primarily target invaders; however, excessive reactive oxygen species can cause injury to host cells and organs. For example, deRojas-Walker et al. (1995) identified oxidation and deamination products in the DNA of activated macrophages exposed to E. coli lipopolysaccharide.

Oxidative stress is widely believed to contribute to the development of diverse age-related diseases and to the aging process in humans. Similarly, whatever the inducer, it has been suggested to impact various economic traits of chickens, like feed efficiency, meat quality, and semen quality. However, the potential effect of oxidative stress on immunocompetence is not well understood. Experiments on immune cells seem to show incompatible effects of oxidative stress, while increasing evidence in animals, like chickens, cows, rats and mice, suggested that elevated oxidative stress impairs immune function (Deng, 2010).

As yet another example in poultry, stress-induced immunosuppression is manifested by failures in vaccination and increased morbidity and mortality of flocks. Currently, several modern cellular and molecular approaches are being used to explore the status of the immune system during stress and disease. It is likely that these new techniques will lead to the development of new strategies for preventing and controlling immunosuppression in poultry (Shini et al., 2010).

One strategy is that the administration of antioxidants improves the immune response. In one study, epigallocatechin-3-gallate (EGCG), a polyphenol derived from green tea, alleviates the heat stress induced oxidative stress in quail via its antioxidant effects. The mechanism of action by which EGCG is alleviating oxidative stress is believed to be through modulating the hepatic nuclear transcription factors in heat-stressed quails (Sahin et al., 2010).

Grape extracts containing antioxidants may also reduce oxidative stress. In one study focusing on γδ T cells, members of the proanthocyanidin family and the anthocyanin family of compounds were tested. Because grapes and grape products contain both of these types of compounds, the hypothesis was that grapes may help maintain or support the immune response, specifically the γδ T cell. Data from intact animal studies show that immune function is supported by grape products. In humans, relatively little research has been conducted using the food as an intervention; however, a study currently in progress showed that grape juice supported circulating γδ T cells and maintained immune function, whereas participants receiving the placebo juice had changes associated with reduced immunity (Percival, 2009).

Antioxidants may also have antigenotoxic and immunomodulatory functions. In a randomized crossover study in healthy men on a low-polyphenol diet, the effects of two polyphenol-rich juices (330 ml/d) supplemented for 2 weeks was determined. Bioavailability of polyphenols, markers of antioxidative and immune status, and reduction of DNA damage were measured. Juices provided 236 mg (A) and 226 mg (B) polyphenols with cyanidin glycosides (A) and epigallocatechin gallate (B) as major polyphenolic ingredients. There was no accumulation of plasma polyphenols after two weeks of juice supplementation. In contrast, plasma malondialdehyde decreased with time during juice interventions. Moreover, juice consumption also increased lymphocyte proliferative responsiveness, with no difference between the two juices. Interleukin-2 secretion by activated lymphocytes and the lytic activity of natural killer cells were significantly increased by both juices. Juice intervention had no effect on single DNA strand breaks, but significantly reduced oxidative DNA damage in lymphocytes. A time-delay was observed between the intake of fruit juice and the reduction of oxidative DNA damage and the increase in interleukin-2 secretion. The conclusion is that consumption of either juice enhanced antioxidant status, reduced oxidative DNA damage and stimulated immune cell functions (Bub et al., 2003).

In another study, the possible protective effect of grape seed extract was investigated against gamma-radiation-induced oxidative stress in heart and pancreas tissues associated with serum metabolic disturbances. Exposure to ionizing radiation induces the formation of reactive oxygen species in different tissues provoking oxidative damage, organ dysfunction, and metabolic disturbances. Irradiated rats were whole body exposed to 5 Gy gamma radiation. Grape seed extract (GSE) treated irradiated rats received 100 mg GSE/kg/day, by gavage, for 14 days before irradiation. The animals were sacrificed on days 1, 14 and 28 after irradiation. Significant decreases of superoxide dismutase, catalase and glutathione peroxidase activities associated with significant increases of thiobarbituric acid reactive substance (TBARS) levels were recorded in heart and pancreas tissues after irradiation. The TBARS were assayed as an indicator of lipid peroxidation. GSE administration pre-irradiation significantly attenuated the radiation-induced oxidative stress in heart tissues which was substantiated by a significant amelioration of serum lactate dehydrogenase, creatine phosphokinase and aspartate transaminase activities. GSE treatment also attenuated the oxidative stress in pancreas tissues which was associated with a significant improvement in radiation-induced hyperglycemia and hyperinsulinemia. In conclusion, the present data demonstrate that GSE would protect the heart and pancreas tissues from oxidative damage induced by ionizing irradiation (Saada et al., 2009).

In addition to grape extract, there is some evidence to suggest that trace elements may also alleviate oxidative stress. In one study, the element selenium dissolved in dimethyl sulfoxide (Se/DMSO) was tested with and without grape seed extract dissolved in saline (GSE/saline) and compared with GSE/saline alone in alleviating gastric lesions caused by indomethacin in rats. GSE/saline and Se/DMSO, alone and in combination, significantly reduced gastric lesions and increased the enzymatic antioxidants superoxide dismutase, catalase and glutathione peroxidase. It is believed that selenium and grape seed extract have a protective effect against indomethacin-induced gastric ulcers through prevention of lipid peroxidation, activation of radical scavenging enzymes and anti-inflammatory activity. Selenium and grape seed extract combined was more effective than either alone (Abbas and Sakr, 2013).

French Patent No. 2940014 discloses an antioxidant composition particularly for use in ruminant animals, containing high concentrations of micronutrients and plant extracts. The composition includes 15-60% zinc amino acid chelate, 2-10% rumen-protected selenium, 3-15% organic selenium, and at least one plant extract such as extract from lemon balm, grape, blueberry, pomegranate, apple, and onion. The composition is incorporated at concentrations of 0.5-6% in a feed supplement, nutritional supplement or mineral feed.

The available evidence indicates that there may be a connection between the antioxidants found in, for example, grape seed extract and a positive immune response. There may further be evidence for trace elements such as selenium in alleviating the effects of lipid peroxidation, activating scavenging enzymes and promoting anti-inflammatory activity. There remains a need, however, to administer the antioxidant properties of these substances easily and effectively to a variety of animals, including poultry, swine, and rabbits, among others.

### SUMMARY OF THE INVENTION

In a first aspect, the invention is a composition containing sodium bicarbonate, grape extract, an organic acid, and selenium, wherein the composition comprises about (a) about 10 percent (w/w) to about 30 percent (w/w) of grape extract; (b) about 30 percent (w/w) to about 60 percent (w/w) of sodium bicarbonate; (c) about 0.01 percent (w/w) to about 0.50 percent (w/w) of selenium; (d) about 0.25 percent (w/w) to about 2.0 percent (w/w) of a lubricant; (e) about 20 percent (w/w) to about 40 percent (w/w) dry organic acid; and (f) optionally, about 1.0 percent (w/w) to about 3.0 percent (w/w) of a binder.

In one embodiment, the selenium is derived from an organic source, such as yeast. In another embodiment, the selenium is derived from an inorganic source, such as sodium selenite.

In a second aspect, the invention is a method of improving the stress response in an animal comprising the step of administering a composition containing sodium bicarbonate, grape extract, organic acid, and selenium followed by the step of administering a stressor. In one embodiment, the step of administering a composition containing sodium bicarbonate, grape extract, organic acid, and selenium includes selenium from an organic source, such as yeast. In another embodiment, the step of administering a composition containing sodium bicarbonate, grape extract, organic acid, and selenium includes selenium from an inorganic source, such as sodium selenite. In yet another embodiment, the step of administering a stressor includes administering a vaccine to an animal.

In a third aspect, the invention is a method for producing meat comprising raising an animal on a diet that is supplemented with a composition according to the first aspect.

### BRIEF DESCRIPTION OF THE DRAWINGS

FIG. 1 depicts the blood concentration of the oxidative stress marker, PROTOX in effervescent Grape/Se treated piglets versus untreated piglets. PROTOX is the concentration of blood plasma protein and lipid oxidative stress markers.

FIG. 2 depicts the ratio PROTOX/CATH in blood from effervescent Grape/Se treated piglets versus untreated piglets. PROTOX is a marker for oxidative processes; CATH is the concentration of hydrophilic total antioxidant capacity.

### DETAILED DESCRIPTION OF THE INVENTION

The invention encompasses a nutritional product and method of using the product in animals with the aim of fighting against exaggerated or increased oxidative process following a stressor, such as may occur during vaccination, weaning, transportation, and environmental challenges.

In one aspect, the invention is a composition containing sodium bicarbonate, organic acid, grape extract, and selenium, wherein the grape extract is about 10 percent (w/w) to about 30 percent (w/w) of the composition; sodium bicarbonate is about 30 percent (w/w) to about 60 percent (w/w) of the composition; the organic acid is dry organic acid which is about 20 percent (w/w) to about 40 percent (w/w) of the composition; and the selenium is about 0.01 percent (w/w) to about 0.5 percent (w/w) of the composition. The composition also includes about 0.25 percent (w/w) to about 2.0 percent (w/w) of a lubricant. In some embodiments, the amount of lubricant is about 0.5 percent (w/w) to about 1.0% (w/w). Lubricants are ingredients added in small amounts to improve processing such as to prevent ingredients from clumping together and to prevent sticking to a tablet making apparatus. Lubricants known in the art of tablet manufacture, such as glyceryl tristearate, stearic acid, and talc may be included in certain embodiments of the invention. In some embodiments of the invention, the lubricant is magnesium stearate.

The instant invention is an effervescent composition, which in one preferred embodiment is in the form of a tablet, alternatively called a disc or puck. The effervescent quality is provided by the presence of sodium bicarbonate and organic acid, which produces carbon dioxide bubbles when the composition is added to water. As would be appreciated by one skilled in the art, sodium bicarbonate and organic acid will have an effect on the pH of the solution that is formed when the tablet is dissolved in water. In addition, the effervescence will aid the dissolution of the tablet due to the turbulence of gas bubble formation. In certain powder or tablet embodiments of the instant invention, the organic acid is dry organic acid. In some embodiments, the organic acid comprises at least one acid selected from group consisting of citric acid, fumaric acid, or tartaric acid. In one embodiment the organic acid preferably comprises citric acid.

The composition optionally contains about 1.0 percent (w/w) to about 3.0 percent (w/w) of a binder. By binder is meant a substance which helps to hold a tablet together. Non-limiting representative binders include acacia, cellulose, gelatin, methyl cellulose, starch, polyvinylpyrrolidone, and polyethylene glycol. Other pharmaceutically acceptable excipients known to those skilled in the art to be suitable for manufacture of tablets are optionally included in some embodiments of the invention. These excipients may be, for example, inert diluents, such as calcium carbonate, sodium carbonate, lactose, calcium phosphate or sodium phosphate; granulating and disintegrating agents, for example, corn starch, or alginic acid. Other additives such as preservatives, colorants, and solubility enhancers, may optionally be included in certain embodiments of the instant invention.

In one embodiment, the selenium is organically derived. In another embodiment, the selenium is inorganic selenium. In yet another embodiment, selenium is a combination of inorganic and organically derived selenium. Organic selenium may be in the form of selenomethione (SeMet). In certain embodiments, the organically derived selenium is from yeast such as a strain of the yeast Saccharomyces cerevisiae grown in a sodium selenite medium. In another embodiment, the selenium is sodium selenite. In another embodiment the inorganic selenium is calcium selenite. In some embodiments, the selenium concentration in the composition is about 0.05% to about 0.20% (w/w). In another embodiment, the concentration of selenium is about 0.01 to about 0.10% (w/w). In one embodiment, the composition preferably contains 506 mg/kg selenium; in another embodiment, the composition preferably contains 0.11% (w/w) sodium selenite.

The minimum nutrient requirement for selenium in animals is typically in the range of 0.1-0.3 mg/kg. Selenosis, toxicity due to selenium, is known to occur when selenium is consumed at significantly higher amounts than the minimum nutrient requirement. The levels of selenium provided by the instant invention are well below levels considered to be toxic. Table 1 shows representative typical daily water consumption levels for several species of animals, and the amount of selenium that would be provided by one exemplary embodiment of the instant invention.

TABLE 1
10 g Effervescent Grape Extract Tablet
(20% Grape Extract, 506 mg/kg selenium,
48.41% sodium bicarbonate, 0.5% magnesium stearate)
mixed with 100 liters of drinking water

| | Selenium | Daily Water Consumption |
|---|---|---|
| Species | (in mg) | (in liters) |
| Rabbit | 0.05 | 0.5 |
| Chicken | 0.005-0.006 | 0.09-0.12 |
| Piglet | 0.15 | 3 |

In some embodiments, the grape extract in the instant invention is a powder obtained by extraction of grape (Vitis vinifera) with water and alcohol. After extraction the grape extract is dried to provide a powder, using a technique such as spray drying. Other methods of drying an extract that are known in the art, including freeze-drying, are suitable for some embodiments. In certain embodiments, the grape extract is preferably rich in polyphenols (>80%) and procyanidols (>60%), and contains anthocyanins (>0.75%).

In another aspect, the invention is a method of improved stress response in an animal comprising the step of administering a composition containing sodium bicarbonate, grape extract, organic acid, and selenium, followed by the step of administering to or subjecting the animal to a stressor. In one embodiment, the composition containing selenium comprises sodium selenite. In one embodiment, the composition containing selenium comprises selenium from a yeast source.

Administration in drinking water is a convenient way to supplement an animal's diet with compositions of the invention. Accordingly, in one embodiment, the method of improved stress response comprises administering a sodium bicarbonate, grape extract, organic acid, and selenium composition to an animal in the drinking water. In one preferred embodiment, the composition comprises

(a) about 10 percent (w/w) to about 30 percent (w/w) of grape extract;
(b) about 30 percent (w/w) to about 60 percent (w/w) of sodium bicarbonate;
(c) about 0.01 percent (w/w) to about 0.50 (w/w) percent of selenium;
(d) about 0.25 percent (w/w) to about 2.0 percent (w/w) of a lubricant;
(e) about 20 percent (w/w) to about 40 percent (w/w) of dry organic acid; and
(f) optionally, about 1.0 percent (w/w) to about 3.0 percent (w/w) of a binder,
and the composition is added to drinking water so that the concentration of the composition in the water is about 0.05 percent (w/v) to about 0.2 percent (w/v).

In some embodiments of the invention, the composition of sodium bicarbonate is about 40 to about 60 percent (w/w). In some embodiments the concentration of lubricant is about 0.50 to about 1.0 percent (w/w).

In one embodiment of the invention, the dry organic acid comprises at least one acid selected from the group consisting of citric acid, fumaric acid, and tartaric acid. In one embodiment, the dry organic acid preferably comprises citric acid.

In one embodiment of the invention, the selenium in the composition is preferably about 0.05 percent (w/w) to about 0.20 percent (w/w). In one embodiment, the concentration of selenium is about 0.01 percent (w/w) to about 0.1 percent (w/w). In one embodiment, the composition preferably contains 506 mg/kg selenium; in another embodiment, the composition preferably contains 0.11% (w/w) sodium selenite.

In one embodiment, administering a stressor to an animal comprises administering a vaccine. In another embodiment, subjecting an animal to a stressor comprises weaning a warm blooded animal, whereby stress is created due to loss of antioxidant defenses provided by the mother's milk. In other embodiments, the step of subjecting an animal to a stressor includes exposure to an environmental challenge, such as exposure to extreme temperatures or transporting the animal. During times of stress, oxidative phenomena in the bodies of the animals may undermine antioxidant defenses, a situation called oxidative stress. In such cases, a means to counteract deleterious effects of oxidative insult, and improve animal health is desirable. The instant invention provides compositions that may be used as a protection or insurance against potential ill effects of stress because it provides a means to augment the antioxidant defenses of an animal.

In one embodiment, a method for improved stress response is provided wherein the step of administering to an animal a composition comprising sodium bicarbonate, grape extract, dry organic acid, and selenium composition improves, i.e. increases, the hydrophilic total antioxidant capacity (CATH). In one embodiment of the invention, the step of administering to an animal a composition containing sodium bicarbonate, grape extract, dry organic acid, and selenium improves, i.e. decreases, the oxidative stress ratio (PROTOX/CATH).

PROTOX and CATH are markers in blood that are used for assessing oxidative stress (Frank Duncombe Laboratory “Analyses De Stress Oxydant”, 2014). CATH is indicative of the level of antioxidant defenses. PROTOX is a marker for oxidative processes. Increased CATH levels are an indication of an improved stress response because antioxidants counteract oxidation processes. Lower PROTOX levels are an indication of lower levels of oxidative processes. A lower PROTOX/CATH ratio equates with an improved stress response because a lower PROTOX/CATH ratio can be an outcome of lowering PROTOX (i.e. oxidative processes), raising CATH (i.e. antioxidant defenses), or a combination of the two.

In one aspect, the instant invention provides a method of producing meat with improved quality comprising raising an animal on a diet that is supplemented with a composition comprising:

(a) about 10 percent (w/w) to about 30 percent (w/w) of grape extract;
(b) about 30 percent (w/w) to about 60 percent (w/w) of sodium bicarbonate;
(c) about 0.01 percent (w/w) to about 0.50 percent (w/w) of selenium;
(d) about 0.25 percent (w/w) to about 2.0 percent (w/w) of a lubricant;
(e) and 20 percent (w/w) to about 40 percent (w/w) of dry organic acid; and
(f) optionally, about 1.0 percent (w/w) to about 3.0 percent (w/w) of a binder.

In some embodiments of the invention, the composition of sodium bicarbonate is about 40 to about 60 percent (w/w). In some embodiments, the concentration of lubricant is about 0.50 to about 1.0 percent (w/w). In some embodiments, the dry organic acid comprises at least one acid selected from the group consisting of citric acid, fumaric acid, and tartaric acid. In one embodiment, the dry organic acid preferably comprises citric acid.

In one embodiment, the concentration of selenium is about 0.05 percent (w/w) to about 0.2 percent (w/w). In one embodiment, the concentration of selenium is about 0.01 percent (w/w) to about 0.1 percent (w/w). In one embodiment, the composition preferably contains 506 mg/kg selenium; in another embodiment, the composition preferably contains 0.11% (w/w) sodium selenite.

In one embodiment of the invention, the composition is administered in drinking water. In another embodiment, the concentration of the composition in the drinking water is preferably about 0.05% (w/v) to about 0.2% (w/v). As would be appreciated by one having general knowledge in the art, the composition may be simply added to the drinking water in one step. Alternatively, it may be preferred, in some cases, to add the composition to a first quantity of water until it is dissolved to form a concentrate, then the concentrate may be diluted by adding it to a reservoir, or second quantity, of water so as to provide drinking water containing the composition at the desired concentration.

Meat producers are motivated to provide a high quality product that meets the demands of the consumer, particularly when they can obtain a higher price for the meat based on a particular quality criterion. For example, the quality of poultry meat is of particular importance to consumers and poultry producers alike, and the qualitative value of the meat is commonly assessed by measuring water loss, pH, meat color, and other sensory characteristics.

In one embodiment of the invention, the quality of the meat is measured by determining the water loss rate, which is decreased in meat from an animal raised on a diet supplemented with the composition as compared to meat from an animal that is raised on a diet that is not supplemented with the composition.

Compositions of the instant invention surprisingly have a significant effect on the water holding capacity of poultry broiler meat. Due to the improved water holding capacity, the water loss rate, also called the exudation rate, is decreased. The rate of water loss may be determined under standard conditions, such as by determining the percentage of moisture lost over time during storage under refrigeration, or during cooking for a period of time. Less moisture loss during cooking is advantageous because it provides a juicer cooked product that is desirable to consumers.

Without being bound by theory, it is believed that the effervescent grape extract/Se compositions according to the invention contribute to better antioxidant status and overall better animal health, resulting in optimized development of the flesh and improved water holding capacity.

### EXAMPLES

Below, the presently disclosed invention will be described by way of examples, which are provided for illustrative purposes only and accordingly are not to be construed as limiting the invention.

### Example 1

### Efficacy in Vaccinated Chickens

The effectiveness of the invention was tested in broiler chicks (Gallus gallus). The composition of the invention was administered six days prior to a stressor event (i.e., vaccination) for four consecutive days and compared with a control. The effect on the consumption of water and food, growth (live weight), feed efficiency, antioxidant status, quality of the meat and mortality was measured.

### General Procedure

### Acclimation Period

All animals (1,680 broilers+spare animals) were housed at a test facility (CEBIPHAR, Fondettes, France; www.cebiphar.com) and examined for any sign of disease or abnormality. The animals were observed daily for signs of any disease throughout the acclimation period. They were individually identified with a unique number using wing clips, weighed and allocated to pens and groups according to Table 1. There were 42 broilers in each cage and each cage was assigned a treatment as explained below.

TABLE 2

| No. of | | | |
|---|---|---|---|
| cage | Treatment | Sex | Area |
| 1 | T4 | M | 1 |
| 2 | T3 | M | 1 |
| 3 | T2 | M | 1 |
| 4 | T1 | M | 1 |
| 5 | T4 | F | 1 |
| 6 | T3 | F | 1 |
| 7 | T2 | F | 1 |
| 8 | T1 | F | 1 |
| 9 | T4 | M | 1 |
| 10 | T3 | M | 1 |
| 11 | T1 | M | 1 |
| 12 | T2 | M | 1 |
| 13 | T3 | F | 1 |
| 14 | T1 | F | 1 |
| 15 | T2 | F | 1 |
| 16 | T4 | F | 1 |
| 17 | T4 | M | 2 |
| 18 | T1 | M | 2 |
| 19 | T2 | M | 2 |
| 20 | T3 | M | 2 |
| 21 | T4 | F | 2 |
| 22 | T1 | F | 2 |
| 23 | T2 | F | 2 |
| 24 | T3 | F | 2 |
| 25 | T4 | M | 2 |
| 26 | T2 | M | 2 |
| 27 | T1 | M | 2 |
| 28 | T3 | M | 2 |
| 29 | T4 | F | 2 |
| 30 | T2 | F | 2 |
| 31 | T1 | F | 2 |
| 32 | T3 | F | 2 |
| 33 | T3 | M | 3 |
| 34 | T1 | M | 3 |
| 35 | T2 | M | 3 |
| 36 | T4 | M | 3 |
| 37 | T3 | F | 3 |
| 38 | T2 | F | 3 |
| 39 | T1 | F | 3 |
| 40 | T4 | F | 3 |

Spare animals were housed in the same building.

### Treatment

The treatment was a composition in a powder form for veterinary use as a supplement added to drinking water. The treatment composition is described in Table 3.

TABLE 3

| | | Quantity |
|---|---|---|
| | | in grams |
| Substance | % w/w | (per 10 grams) |
| Sodium selenite | 0.11% | 0.011 g |
| (46% selenium)¹ | | |
| Grape extract | 20% | 2.000 g |
| Sodium bicarbonate | 48.20% | 4.820 g |
| Citric acid | 30.85% | 3.085 g |
| Magnesium stearate | 0.84% | 0.084 g |
| Total | 100.00% | 10.000 g |

Note
¹46% is the percentage that is pure selenium

The treatment composition was administered daily in the drinking water from D6 (day 6) to D10 (day 10) at a dose as described in Table 4.

TABLE 4

| Lot Description | Dosage |
|---|---|
| Lot 1 (T1) | 10 grams/100 liters water |
| Lot 2 (T2) | 10 grams/50 liters water |
| Lot 3 (T3) | 10 grams/200 liters water |
| Lot 4 (T4) | 0 grams (control) |

The supplemented water in each drinker was refreshed on each treatment day and fresh solution containing the treatment composition was prepared each day of treatment before administration to the animals. Treatment was given approximately at the same time in the morning each day throughout the study.

### Application of a Stressor (Vaccination)

Two stressors in the form of vaccines were used. The first was Gallivac IB88 vaccine for infectious bronchitis administered via nebulization to the 1,680 test subject broilers on D13. The second was HipraGumboro G97 vaccine administered via drinking water to the 1,680 test subject broilers at D17. Both vaccines were administered according to manufacturer's instructions.

### Clinical Observations

The animals were clinically observed daily for signs of illness throughout the study.

### Water Intake

Water consumption of each of the subgroups was recorded every day for two weeks to include the week prior to the vaccination and the week following vaccination.

### Feed Intake

Feed was weighed on a calibrated scale and values recorded on a specific data capture form. Cumulative consumption of food was recorded at D6, D17 and D35.

### Body Weight

Body weight (collective as per cage) was recorded on D0, D6, D17, and D35.

### Blood Collection

Six percent of the tested animals were sampled for blood from a wing vein. The first sample was collected at arrival to obtain a baseline reading of the oxidative stress indicators (see below). Second and third samples were taken on D20 and D35.

Blood was collected into 2 mL tubes from the wing vena of the animals in compliance with CEBIPHAR standard operating procedures. For each sampled animal, 1 EDTA tube and 1 heparinated tube filled up to 1 ml each was used (i.e., 2 ml collected in total per animal). Blood was collected by blood-letting (sacrifice of the chicken) if needed. All tubes were labeled with the CEBIPHAR study number, the animal number, the group, the day of collection and the content. Caution was taken to reduce light exposure of the samples. The tubes were not processed prior to being tested (i.e., no centrifugation, no pooling of samples). No treatments other than indicated in the study plan were administered during the course of the study. Blood samples were shipped to a testing organization within 24 hours in a refrigerated container for analysis.

### End of the Study

Field tests ended at the last weighing and bloodletting of the broilers before slaughter. Fifty two broilers were slaughtered in a slaughterhouse approved for analysis of breast filets and legs in the laboratory, and analysis was conducted on the exudate (water loss), pH and color.

The exudate was measured as follows. The pectoralis major (PM) muscle is weighed (Pi=initial weight), placed in a plastic bag zipper and suspended by a hook (planted in the upper part of the muscle) for seven days at +2 to +4° C. The PM muscles was then wiped dry with paper and weighed again (Pf=final weight after exudation) in order to assess the amount of water lost during the storage of the product. The losses are expressed in percentage of the initial weight.

The pH of breasts filets was analyzed the day after slaughter. The color analysis of the breast filets was conducted seven days after slaughter.

### Results

Forty pens were prepared to accommodate 42 chicks each (minimum surface area 2.37 square meters). The chicks were sexed upon arrival and distributed to the pens. Each pen contained one round trough and one hanging feeder.

The forty pens were divided into four treatment groups as outlined above. The number of males and females for each treatment group was equalized.

### Effect on Growth (Live Weight)

There is a significant treatment effect for male birds only as measured at D6 and D35 of age shown in Table 5.

TABLE 5

| | D6 Weight | D35 Weight |
|---|---|---|
| Treatment | (grams) | (grams) |
| 1 (10 g/100 L) | 162 | 2376 |
| 2 (10 g/50 L) | 165 | 2366 |
| 3 (10 g/200 L) | 168 | 2419 |
| 4 (control) | 168 | 2444 |

*p = 0.05 at D6 and p = 0.001 at D35.

### Effect on Meat Quality

There is also a significant treatment effect in the meat quality as measured by exudation of the breast filets shown in Table 6.

TABLE 6

| | Exudation of |
|---|---|
| Treatment | breast filets |
| 1 (10 g/100 L) | 3.24% |
| 2 (10 g/50 L) | 3.86% |
| 3 (10 g/200 L) | 4.74% |
| 4 (control) | 4.08% |

*p = 0.008

### Antioxidant Status

Several antioxidant related parameters were measured to include hydrophilic total antioxidant capacity (CATH), lipophilic total antioxidant capacity (CATL), glutathione peroxidase on whole blood (GPX), superoxide dismutase (SOD), peroxides plasma lipids (POOL), protein plasma lipids (PROTOX) and vitamin E. The antioxidant related parameters in blood samples were measured using a battery of oxidative stress assays (Analyses De Stress Oxydant (Oxidative Stress Analysis) Laboratory Frank Duncombe. http://www.labo-frank-duncombe.fr/). At 20 days of age, a significant dosage effect was measured for CATH at the grouped high dosage administration (i.e., Lot 1+Lot 2=946.9 μmol) compared with the grouped low dosage administration (i.e., Lot 3+Lot 4=843.4 μmol; *p=0.005). No other parameters measured showed a significant treatment effect.

Other parameters measured but displaying no significant treatment effect included food consumption, water consumption, mortality, pH and color of the breast filets.

### Example 2

### Efficacy in Piglets During Weaning

Oxidative stress may be created in mammals at weaning due to withdrawal of the antioxidants received from the mother's milk. The effect of treatment of piglets with an effervescent grape/Se formulation was investigated to determine whether the formulation counteracted the loss of antioxidants at weaning. The study was designed to assess whether there was any quantifiable mitigating effect of the grape/Se formulation upon losing the mother's milk antioxidant defenses.

The treated group of piglets (n=15) consumed drinking water for 5 days that was treated with a Grape/Se effervescent tablet; the control group of piglets (n=15) consumed drinking water without any treatment. The composition of a 100 g Grape/Se effervescent tablet that was added to the drinking water of the treated group is shown in Table 7. The tablet was mixed with 100 liters of drinking water.

TABLE 7

| | | Quantity in grams |
|---|---|---|
| Substance | % w/w | (per 100 gram tablet) |
| Sodium selenite (46%)¹ | 0.0506% | 0.0506 g |
| Grape extract | 20% | 20.00 g |
| Sodium bicarbonate | 48.20% | 48.20 g |
| Citric acid | 30.85% | 30.85 g |
| Magnesium stearate | 0.84% | 0.84 g |

Note
¹46% is the percentage that is pure selenium

There was no significant difference at 20 days or 39 days after weaning between the weights of the piglets in the control groups as compared to the treated test group. However, there was a significant increase at 20 days after weaning in the concentration of hydrophilic total antioxidant capacity (CATH) in the treated group as compared to the control group.

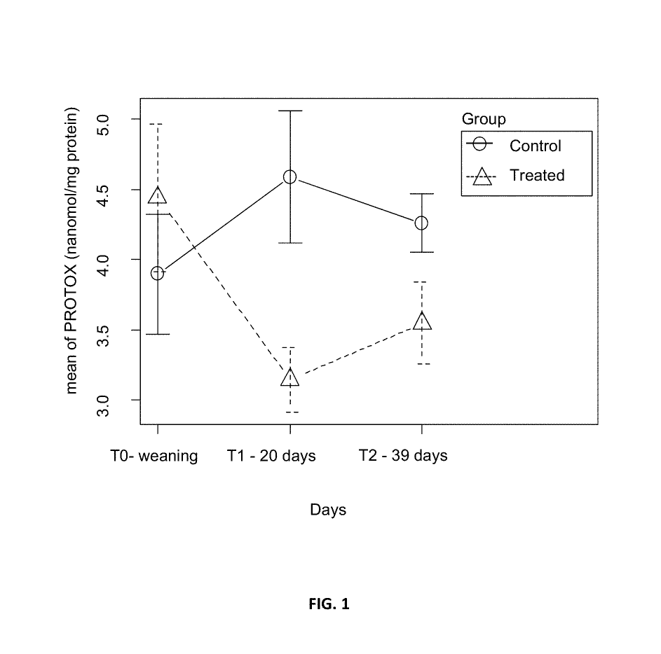

In addition, as depicted in FIG. 1, the concentrations of proteins and lipids that are markers of oxidative processes (PROTOX) are significantly decreased in the treated group at days 20 and 39 after weaning, as compared to the control group. The PROTOX increase in the control group was concurrent with withdrawal of the mother's milk; however the blood concentrations of the PROTOX stress markers was comparatively decreased in the group that received the effervescent Grape/Se composition.

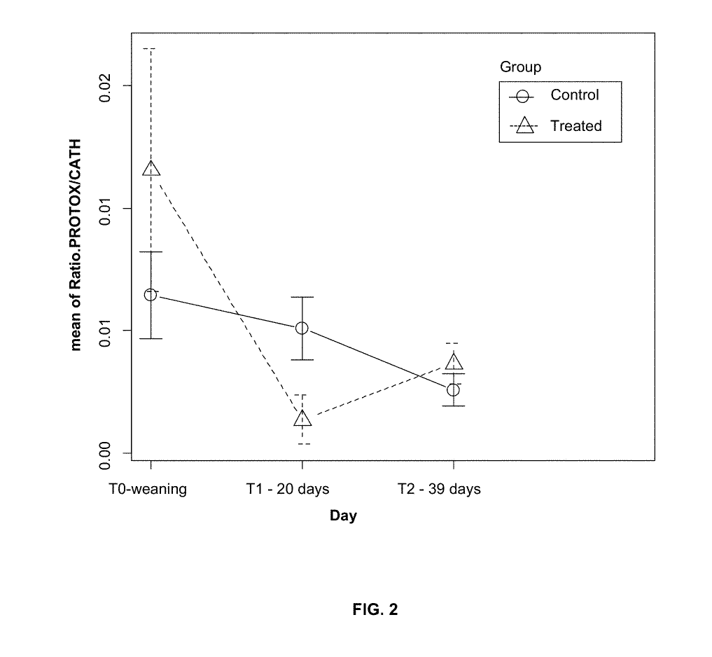

FIG. 2 depicts the ratio of the oxidative stress marker, PROTOX, relative to the hydrophilic total antioxidant capacity, CATH. At day 20 after weaning, the ratio PROTOX/CATH is significantly lower in the treated group than the control group, demonstrating that there is a correlation between the drinking water treatment and an improvement to the oxidative balance in the treated piglets. This effect is surprising in view of the fact that the treatment of the piglets was for a short duration of only 5 days after weaning, and the level of Se is in the low end of typical nutritional Se supplementation in animals.

In conclusion, these studies show that the effervescent grape/Se tablet according to the instant invention is effective in providing a quantifiable reduction of oxidative stress in weanling piglets.

### REFERENCES

Abbas A M, Sakr H F. Effect of selenium and grape seed extract on indomethacin-induced gastric ulcers in rats. J Physiol Biochem 2013; 69:527-537.
Bub A, Watzl B, Blockhaus M, Briviba K, Liegibel U, Müller H, Pool-Zobel B L, Rechkemmer G. Fruit juice consumption modulates antioxidative status, immune status and DNA damage. J Nutr Biochem 2003; 14(2):90-8.
Clapp B R, Hingorani A D, Kharbanda R K, Mohamed-Ali V, Stephens J W, Vallance P, MacAllister R J. Inflammation-induced endothelial dysfunction involves reduced nitric oxide bioavailability and increased oxidant stress. Cardiovascular Research 2004; 64:172-178.
Constantini D, Dell'Omo G. Effects of T-cell-mediated immune response on avian oxidative stress. Comparative Biochemistry and Physiology, Part A 2006; 145:137-142.
Deng H. An assessment of the relationship among oxidative stress, adaptive immunity and genetic variations in the chicken, Gallus gallus. Ph.D. Dissertation, Virginia Polytechnic Institute and State University, Blacksburg, Va., Sep. 1, 2010.
deRojas-Walker T, Tamir S, Ji H, Wishnok J S, Tannebaum S R. Nitric oxide induces oxidative damage in addition to deamination in macrophage DNA. Chem Res Toxicol 1995; 8:473-477.
Frank Duncombe Laboratory. Analyses De Stress Oxydant. 2014, p. 15.
Keles H, Fidan A F, Cigerci I H, Kucukkurt I, Karadas E, Dundar Y. Increased DNA damage and oxidative stress in chickens with Natural Marek's Disease. Veterinary Immunology and Immunopathology 2010; 133:51-58.
Percival S. Grape consumption supports immunity in animals and humans. J Nutr 2009; 139: 1801S-1805S.
Saada H N, Said U Z, Meky N H, Abd El Azime A S. Grape seed extract Vitis vinifera protects against radiation-induced oxidative damage and metabolic disorders in rats. Phytother Res 2009; 23(3):434-8.
Sahin K, Orhan C, Tuzcu M, Ali S, Sahin N, Hayirli A. Epigallocatechin-3-gallate prevents lipid peroxidation and enhances antioxidant defense system via modulating hepatic nuclear transcription factors in heat-stressed quails. Poultry Science 2010; 89:2251-2258.
Shini S, Huff G R, Shini A, Kaiser P. Understanding stress-induced immunosuppression: Exploration of cytokine and chemokine gene profiles in chicken peripheral leukocytes. Poultry Science 2010; 89:841-851.

1. A composition comprising:
(a) about 10 percent (w/w) to about 30 percent (w/w) of grape extract;
(b) about 30 percent (w/w) to about 60 percent (w/w) of sodium bicarbonate;
(c) about 0.01 percent (w/w) to about 0.50 percent (w/w) of selenium;
(d) about 0.25 percent (w/w) to about 2.0 percent (w/w) of a lubricant;
(e) about 20 percent (w/w) to about 40 percent (w/w) dry organic acid; and
(f) optionally, about 1.0 percent (w/w) to about 3.0 percent (w/w) of a binder.

2. The composition of claim 1 wherein the selenium is organically derived or is a sodium selenite salt.

3. The composition of claim 2 wherein the selenium is present in an amount of about 0.05 percent (w/w) to about 0.20 percent (w/w).

4. The composition of claim 2 wherein the selenium is present in an amount of about 0.01 percent (w/w) to about 0.1 percent (w/w).

5. A method for improved stress response comprising the step of administering to an animal a composition containing sodium bicarbonate, grape extract, dry organic acid, and selenium, followed by the step of administering to or subjecting the animal to a stressor.

6. A method for improved stress response according to claim 5, wherein the composition containing selenium comprises sodium selenite or selenium from a yeast source, or a combination thereof.

7. A method for improved stress response according to claim 5, wherein the composition is administered in drinking water.

8. A method for improved stress response according to claim 5, wherein the step of administering a stressor comprises administering a vaccine.

9. A method for improved stress response according to claim 5, wherein the step of administering a composition containing sodium bicarbonate, grape extract, dry organic acid, and selenium improves the hydrophilic total antioxidant capacity (CATH).

10. A method of improved stress response according to claim 5, wherein administering to an animal a composition containing sodium bicarbonate, grape extract, dry organic acid, and selenium improves the oxidative stress ratio (PROTOX/CATH).

11. A method for improved stress response according to claim 5, wherein the step of subjecting the animal to a stressor comprises weaning, exposure to an environmental challenge, or transportation.

12. The method of improved stress response according to claim 11, wherein the step of subjecting the animal to a stressor is weaning.

13. A method of improved stress response according to claim 5, wherein said composition comprises
(a) about 10 percent (w/w) to about 30 percent (w/w) of grape extract;
(b) about 30 percent (w/w) to about 60 percent (w/w) of sodium bicarbonate;
(c) about 0.01 percent (w/w) to about 0.50 percent (w/w) of selenium;
(d) about 0.25 percent (w/w) to about 2.0 percent (w/w) of a lubricant;
(e) about 20 percent (w/w) to about 40 percent (w/w) of dry organic acid; and
(e) optionally, about 1.0 percent (w/w) to about 3.0 percent (w/w) of a binder;
wherein the step of administering the composition comprises providing drinking water that contains about 0.05% (w/v) to about 0.2% (w/v) of said composition.

14. The method of improved stress response according to claim 13, wherein the dry organic acid comprises at least one acid selected from the group consisting of citric acid, fumaric acid, and tartaric acid.

15. The method of improved stress response according to claim 13, wherein the drinking water containing the composition is provided to the animal for a period of time that is effective to improve the oxidative stress ratio (PROTOX/CATH).

16. The method of improved stress response according to claim 13, wherein the selenium in the composition is present in an amount of about 0.05 percent (w/w) to about 0.20 percent (w/w).

17. A method of producing meat with improved quality comprising raising an animal on a diet that is supplemented with a composition comprising:
(a) about 10 percent (w/w) to about 30 percent (w/w) of grape extract;
(b) about 30 percent (w/w) to about 60 percent (w/w) of sodium bicarbonate;
(c) about 0.01 percent (w/w) to about 0.50 percent (w/w) of selenium;
(d) about 0.25 percent (w/w) to about 2.0 percent (w/w) of a lubricant;
(e) about 20 percent (w/w) to about 40 percent (w/w) of dry organic acid; and
(f) optionally, about 1.0 percent (w/w) to about 3.0 percent (w/w) of a binder.

18. A method of producing meat with improved quality according to claim 17, wherein said composition is administered in drinking water.

19. A method of producing meat with improved quality according to claim 18, wherein the concentration of the composition in drinking water is about 0.05 percent (w/v) to about 0.2 percent (w/v).

20. A method of producing meat with improved quality according to claim 17, wherein the quality of the meat is measured by determining the water loss rate, which is decreased in meat from an animal raised on a diet supplemented with the composition as compared to meat from an animal that is raised on a diet that is not supplemented with the composition.
